(19) United States
(12) Patent Application Publication
(10) Pub. No.: US 2026/0257041 A1
(43) Pub. Date: Sep. 3, 2026

(54) ARTICULATION OF A CHRONIC TOTAL OCCLUSION (CTO) CROSSING DEVICE TO AN OPTIMAL ENTRY POINT

(71) Applicant: KONINKLIJKE PHILIPS N.V., EINDHOVEN (NL)

(72) Inventors: Leili Salehi, WALTHAM, MA (US); René Leonardus Jacobus Marie Ubachs, EINDHOVEN (NL); Vipul Shrihari Pai Raikar, SOMERVILLE, MA (US); Alyssa Torjesen, EXETER, NH (US); Bernardus Hendrikus Wilhelmus Hendriks, EINDHOVEN (NL)

(21) Appl. No.: 19/164,623

(22) PCT Filed: Apr. 4, 2024
(86) PCT No.: PCT/EP2024/055525

§ 371 Date: Sep. 12, 2025

Related U.S. Application Data
(60) Provisional application No. 63/451,683, filed on Mar. 13, 2023.

(51) Int. Cl. A61M 25/01; A61M 25/00; A61M 25/09

(52) U.S. Cl. CPC A61M 25/0158; A61M 25/005; A61M 25/09

(57) ABSTRACT

An intravascular therapy device (10) includes a catheter (12) having a guidewire lumen (16) configured to pass a guidewire (18) therethrough; and at least one arm (32) branching away from a distal end of the catheter at a nonzero angle respective to a central axis of the catheter. The at least one arm is configured to steer a tip of the guidewire away from the central axis of the catheter as the tip of the guidewire exits the guidewire lumen at the distal end of the catheter.

### FIELD

The following relates generally to the catheter arts, mechanical intravascular arts, device articulation arts, and related arts.

### BACKGROUND

A chronic total occlusion (CTO) is a complete obstruction of a blood vessel. To treat such a CTO (or other nearly complete occlusion) in a typical intervascular therapy workflow, a guidewire is initially inserted into the blood vessel and manipulated to cross the occlusion, which can be composed of fibrotic or calcific materials, for example. Additionally, in the case of an occlusion due to a thrombus or thromboemboli (sometimes also referred to herein as a clot), it may also be necessary to cross the occlusion. After crossing the occlusion, an interventional catheter (i.e., a balloon/stent device, etc.) is inserted along the guidewire to access the CTO to perform the treatment. The interventional catheter includes one or more tools to perform the treatment, such as a mechanical cutter, a laser aperture for laser cutting, a balloon to perform angioplasty, various combinations thereof, and/or so forth. The interventional catheter may also, optionally, include other features such as an aspiration lumen for removing debris produced by the treatment.

When attempting to drive the guidewire to cross an obstruction, one approach is to pass through the CTO or other obstruction. However, this may be difficult or impossible, as the CTO is (at least nearly) a complete obstruction and may be calcified or otherwise too hard for the guidewire to penetrate. Passing hydrophilic wires and catheters in a subintimal plane of the occluded vessel is an alternative way of crossing the CTO in the case of a hard occlusion. According to one report, technical success for subintimal angioplasty (SIA) is 85.7% (M. J. Bown, A. Bolia, and A. J. Sutton, “Subintimal Angioplasty: Meta-analytical Evidence of Clinical Utility,” Eur. J. Vasc. Endovasc. Surg., vol. 38, no. 3, pp. 323-337, September 2009, doi: 10.1016/j.ejvs.2009.05.014). According to that report the success rate is limited primarily by failure of re-entry to the true lumen, inaccuracy of re-entry, and increasing risk of complications. In guidewire crossing via the subintimal space, the guidewire passes along the blood vessel wall, thus, presenting risks such as the guidewire penetrating through the vessel wall, or exiting through a branching blood vessel without completing the crossing, so that the guidewire does not re-enter the blood vessel lumen after completely crossing the occlusion. Entering the subintimal layer from an optimal direction with the guidewire directed close to parallel with the wall of the blood vessel can increase the likelihood of an optimal true lumen re-entry. In some cases, the position of the subintimal crossing around the circumference of the blood vessel lumen may also impact the likelihood of success. For example, if there is a branching vessel located at the occlusion, then a subintimal crossing 90 degrees away from that branch around the vessel lumen will be more likely to be successful than an attempted subintimal crossing at the circumferential position of the branching vessel, since in the latter case the guidewire has a substantial possibility of entering the branching vessel rather than completing the crossing of the occlusion. An angioplasty in this location can result in a higher chance of revascularization of the side branch. Similarly, if there is a priori knowledge of a lesion or other damaged vessel wall section, the subintimal crossing is preferably performed away from that damaged vessel region. The optimal entry point can be detected by analyzing information about the vessel morphology and the stenosis morphology and composition obtained by ultrasound and/or computed tomography (CT) imaging and/or other medical diagnostics. Even knowing the entry point, however, it is still difficult to accurately articulate the guidewire or other crossing device to it.

Lack of control of the point of entry to the subintimal area may result in various complications such as re-entry inside the stenosis, outside the vessel, the side branches, etc. which can lead to the additional procedure time or patient risk. Moreover, in case of presence of any side branch along the stenosis, a suboptimal crossing path can result in the occlusion of the side branch after balloon/stent angioplasty procedure.

The following discloses certain improvements to overcome these problems and others.

### SUMMARY

In some embodiments disclosed herein, an intravascular therapy device includes a catheter having a guidewire lumen configured to pass a guidewire therethrough; and at least one arm branching away from a distal end of the catheter at a nonzero angle respective to a central axis of the catheter. The at least one arm is configured to steer a tip of the guidewire away from the central axis of the catheter as the tip of the guidewire exits the guidewire lumen at the distal end of the catheter.

In some embodiments disclosed herein, an intravascular therapy method includes passing a guidewire through a guidewire lumen of a catheter; and using at least one arm of a device, steering a tip of the guidewire away from a central axis of the catheter as the tip of the guidewire exits the guidewire lumen at a distal end of the catheter.

One advantage resides in providing a blood vessel asymmetric entry device that can articulate a guidewire to a desired entry point around the vessel circumference.

Another advantage resides in facilitating a controlled entry for subintimal crossing during an intravascular procedure.

Another advantage resides in providing steerable entry of a guidewire during an intravascular procedure.

Another advantage resides in providing a blood vessel asymmetric entry device with magnetically-controlled feature for articulating a tip of the entry device.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an embodiment of an intravascular therapy device in accordance with the present disclosure.

FIGS. 2-7 show other embodiments of the device FIG. 1.

### DETAILED DESCRIPTION

In embodiments disclosed herein, an asymmetric entry device is provided for use in performing a crossing of a vascular occlusion, which can be detected in two-dimensional (2D) angiography images, CT images, ultrasound images, or so forth. The orientation of the asymmetric entry device can be determined by markers detectable in the images, or using the asymmetry, and has a hollow arm that the guidewire can pass through and arrive at the desired entry point (or, in other embodiments, the arm may not be hollow and instead the guidewire is guided along the arm). The term “guidewire” as used herein refers to the intravascular crossing device used to cross a CTO or other occlusion. The guidewire may be a solid wire, a hollow tube, or narrow-diameter catheter (e.g. microcatheter), or other intravascular instrument suitable for performing the crossing of the occlusion. The asymmetric entry devices disclosed herein find particular application in performing subintimal crossing of an occlusion as they are well-suited for directing the guidewire into a position along a blood vessel wall with the guidewire approximately parallel with that wall. However, the asymmetric entry devices disclosed herein may also find application in other types of occlusion crossing procedures, such as crossing through the occlusion itself (rather than via a subintimal pathway). For example, embodiments of the disclosed asymmetric entry devices could be sized to enable steering of the guidewire to enter into the occlusion at a particular location of the occlusion, for example, with the particular location chosen based on a priori knowledge or estimation from imaging or other diagnostics of a lower thickness and/or reduced hardness of the occlusion at that location.

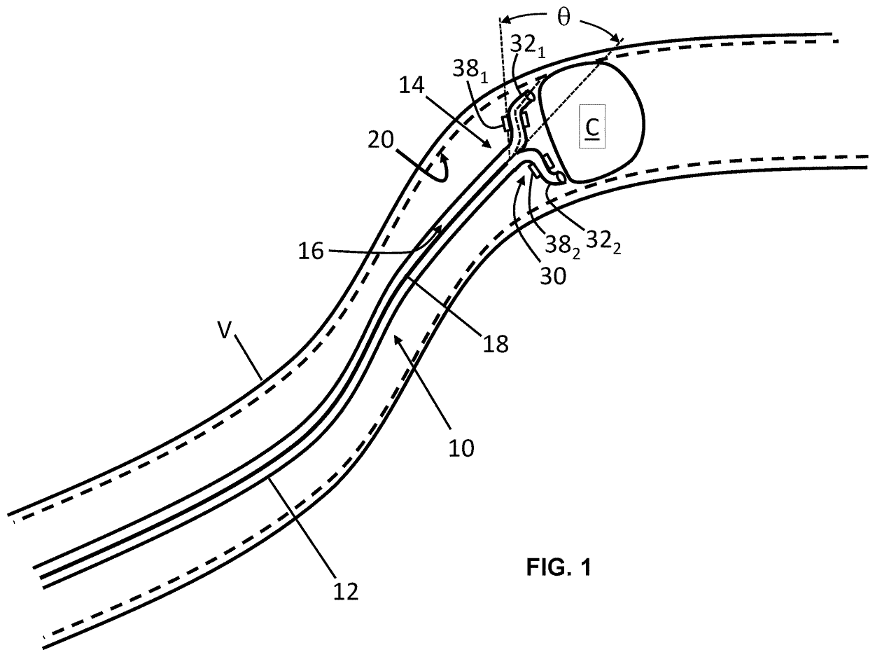

With reference to FIG. 1, an intravascular therapy device 10 for treating a clot C or other occlusion (e.g., a CTO or nearly complete occlusion, or plaque) in a blood vessel V is diagrammatically shown. As used herein, “clot” and “occlusion” refer to a complete, or nearly complete, blockage of the flow of blood through a blood vessel. As shown in FIG. 1, the device 10 includes an interventional device or instrument 12 (e.g., a catheter, and so forth) configured for insertion into a portion of anatomy of a patient, such as into the blood vessel V containing a target such as the occlusion or clot C or so forth. As seen in FIG. 1, the catheter 12 is flexible so that it can follow the contours of the blood vessel V as the catheter is inserted. In a typical intravascular or endovascular procedure, the surgeon or other operator accesses a target by creating an incision (not shown) and inserting a tip 14 of the catheter 12 into a blood vessel V via the incision, and then pushing the interventional instrument 12 into and through the blood vessel V until the tip 14 reaches the target (here the clot C, as shown in FIG. 1). In some examples, the device 10 may include a navigation device with a deployment/retrieval mechanism (not shown), or can reach the clot C using a sheath or a larger catheter than the catheter 12 since the shape of the tip may cause damage to the vessel V. The tip 14 can be flexible enough to spread out when exiting the catheter 12 and obtain the size of the vessel V. The interventional instrument 12 may be radiopaque, at least to the extent that a portion of the tip 14 is visible (potentially with low contrast) in X-ray imaging, ultrasound imaging, or another imaging modality used to monitor the intravascular procedure. The catheter 12, optionally, has a coating of a radiopaque material disposed on the tip 14, or may comprise an attached radiopaque ring made of, for example, platinum or Nitinol wire that is metallurgically bonded (e.g., by welding) to the tip 14 of the interventional instrument 12. These are merely illustrative examples.

FIG. 1 also shows that the catheter 12 includes a guidewire lumen 16 configured to pass a guidewire 18 therethrough. An imaging device (not shown) may be configured to acquire single-shot images and/or a time sequence of images or imaging frames of a position, or movement, of the interventional instrument 12 (and more particularly, a distal portion of the instrument 12 including the radiopaque tip 14). In particular, the imaging device may be a fluoroscopic imaging device (e.g., an X-ray imaging device, C-arm imaging device, a CT scanner, or so forth) and the interventional instrument 12 is visible under the fluoroscopic imaging. The fluoroscopic imaging may be real-time imaging, e.g., with images being acquired at a frame rate of 15-60 frames/second (i.e., 15-60 fps), in some nonlimiting illustrative embodiments. The imaging device used to monitor the intravascular procedure may, for example, comprise an X-ray imaging device such as a C-arm imaging device; however, it will be appreciated that any suitable imaging device, such as ultrasound (US), computed tomography (CT), flat-panel X-ray or fluoroscope, magnetic resonance imaging (MRI), or any other suitable imaging device may be used.

FIG. 1 illustrates an example of a crossing of the occlusion C by the guidewire 18, in which the crossing process is a subintimal crossing. FIG. 1 diagrammatically depicts the subintimal layer 20 of the blood vessel V as a dashed line. The subintimal space between the occlusion C and the main body of the wall of the blood vessel V enables the guidewire to effectively pass “around” the occlusion C, rather than passing through the occlusion. This type of crossing can enable the intravascular procedure to be performed in cases in which the occlusion C is calcified or otherwise hardened so that it is not practical to pass the guidewire 18 through the occlusion C itself. However, a problem can arise in that the interventional instrument 12 is controlled by the physician or other operator via handling performed at the proximal end of the interventional instrument 12, that is, at the end of the catheter or other interventional instrument 12 opposite from the distal end, i.e. tip 14. It can therefore be difficult for the operator to move the guidewire 18 in such a way that it exits from the end of the catheter 12 near the blood vessel wall and is positioned approximately parallel therewith so as to easily enter the subintimal space. To address this problem, as further shown in FIG. 1, an entry device 30 is disposed at the tip 14 of the catheter 12. The entry device 30 is configured to manipulate a position of a tip of the guidewire 18 relative to a wall of the blood vessel V. For example, a tip of the guidewire 18 can be magnetic (e.g. made from steel) or magnetized (e.g. comprising a permanent magnet).

With continuing reference to FIG. 1, the illustrative entry device 30 includes at least one arm, and, in the illustrative example, two arms 32₁ and 32₂. Each arm 32₁ and 32₂ branches away from a distal end of the catheter 12 at a nonzero angle θ respective to a central axis of the catheter 12. (FIG. 1 indicates the angle θ of the first arm 32₁). The branched arm(s) guide the guidewire as the guidewire exits the lumen 16 of the catheter 12 toward the subintimal space of the wall of the blood vessel V. This assists in performing the subintimal crossing of the occlusion C. In some embodiments, the nonzero angle θ is at least 30 degrees, although other angles are contemplated, and the optimal angle can be chosen based on factors, such as the desired maximum length of the entry device 30 (reducing the angle θ may implicate a longer entry device), the diameter of the blood vessel V, the flexibility of the guidewire 18 (the angle θ should be small enough so that the tip of the guidewire 18 can make the turn into the arm), and so forth.

In the illustrative embodiment of FIG. 1, each arm 32₁ and 32₂ has a lumen connected with the lumen 16 of the catheter 12 such that the tip of the guidewire 18 can pass out the end of the lumen 16 of the catheter 12 into the lumen of one of the arms 32₁ or 32₂. This passage is diagrammatically shown in FIG. 1 by a dashed extension of the guidewire 18 passing into and through the lumen of the arm 32₁. As seen in FIG. 1, each arm 32₁ and 32₂ may optionally include a bent tip that is operative to turn the tip of the guidewire 18 as the guidewire exits the lumen of the arm into a path that is approximately parallel with the wall of blood vessel V, thus, further facilitating the subintimal crossing.

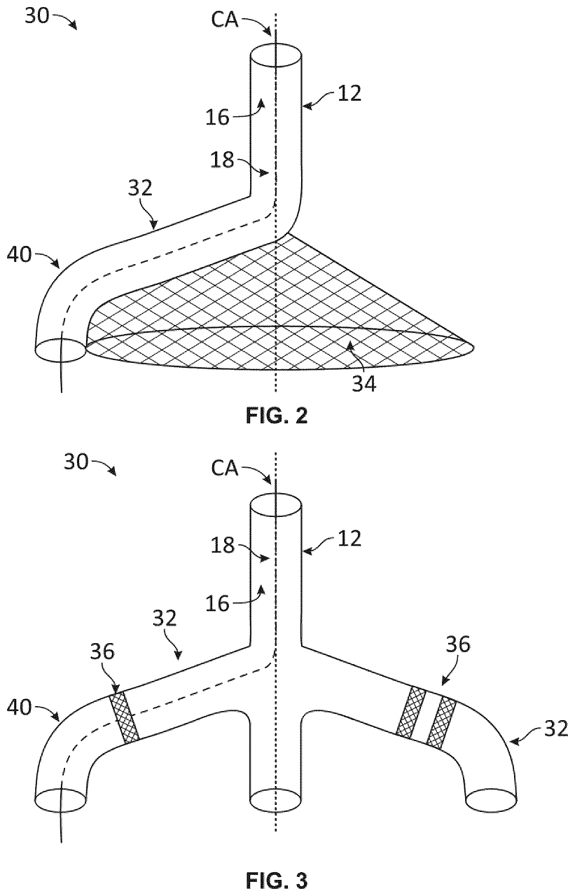

In some embodiments, there may be only a single arm 32 (see illustrative embodiments of FIGS. 2 and 5). In this case, the circumferential position of the subintimal crossing entry around the circumference of the lumen of the blood vessel V can be controlled by the operator rotating the catheter 12 so as to thereby rotate the entry device 30. In other embodiments (e.g., as in the illustrative examples of FIGS. 1, 3, 4, and 6), there may be multiple arms at different circumferential positions around the axis of the catheter 12. In such case, the operator can select which arm to use to guide the subintimal entry for the crossing of the occlusion C. To make the selection, the arms include one or more electromagnets, and the tip of the guidewire 18 is suitably magnetic (e.g., steel) or magnetized (e.g., a permanent magnet). FIG. 1 shows an electromagnet 38₁ disposed on the arm 32₁ and an electromagnet 38₂ disposed on the arm 32₂. The electromagnets are selectively electrically energized (e.g., via electrical leads to the respective electromagnets 38₁ and 38₂ passing through the catheter 12 to an external electrical power supply) to attract the catheter tip into the selected one of the two arms 32₁ or 32₂. By way of one example, in FIG. 1, the electromagnet 38₁ may be energized to create a magnetic field, while the electromagnet 38₂ may not be energized, so that the magnetic (e.g. steel) tip of the catheter 18 is attracted to the energized electromagnet 38₁ and, thus, is steered into the arm 32₁ as shown. (Conversely, if the electromagnet 38₁ is not energized while the electromagnet 38₂ is energized, then the magnetic tip of the catheter 18 will be attracted to the energized electromagnet 38₂ and, thus, is steered into the arm 32₂).

FIGS. 2-6 show further, different embodiments of the entry device 30. As shown in FIG. 2, the entry device 30 comprises a single arm 32, which is hollow and has a lumen that is connected with the lumen 16 at the distal end of the catheter 12. During a subintimal crossing entry, the arm 32 serves to guide the distal end of the guidewire 18 toward the blood vessel wall. The arm 32 branches away from a distal end of the catheter 12 at a nonzero angle respective to a central axis of the catheter 12 (which shown in FIG. 2 with a dashed line CA). For example, the arm 32 branches away from the distal end of the catheter 12 at the nonzero angle respective to the central axis of the catheter of at least 30 degrees, in some nonlimiting illustrative examples. The arm 32 may, in some embodiments, be elastic and configured to be stowed in the distal end of the catheter 12 during delivery into the blood vessel V. For example, the arm 32 may be a nitinol shape-memory tube, whose unconstrained shape is that shown in FIG. 2, and may be compacted into a lumen or other storage recess of the catheter 12 during delivery and, then, retracted back into the recess for withdrawal after the occlusion has been treated. In another example, the arm 32 may be an elastic biocompatible plastic material that, similarly, has the unconstrained shape shown in FIG. 2, and may be compacted into the recess for the delivery and retrieval phases. (While this is described for FIG. 2, it will be appreciated that the arm(s) 32 of any of the embodiments of FIGS. 1-6 may be similarly elastic so as to facilitate delivery and retrieval). The guidewire 18 extends into the blood vessel V after exiting the arm 32 and at this exit is advantageously positioned near the vessel wall to implement a subintimal crossing entry. The illustrative arm 32 is also shown to have a bent tip 40, which is already described with reference to FIG. 1, that advantageously turns the tip of the guidewire 18 as the guidewire exits the lumen of the arm 32 to a position approximately parallel with the blood vessel wall, which further facilitates the subintimal crossing entry. The entry device 30 may include a mesh 34 configured to push the arm 32 outward into the desired angle respective to the central axis CA. That is, the mesh 34 is attached to the arm 32 and configured to expand to bias the arm 32 into the position branching away from the distal end of the catheter 12 at the nonzero angle respective to the central axis CA of the catheter 12. In some examples, in lieu of the mesh 34, the entry device 30 can include any other suitable structure (i.e., springs or a folding mechanism) that could push the arm 32 outward towards the wall of the vessel V.

FIG. 3 shows an embodiment of the entry device 30 that includes multiple arms 32 (three of which are shown in FIG. 3). The arms 32 may include at least one radiopaque marker 36 for visualization of the guidewire 18 in the blood vessel V using a suitable medical imaging device (e.g., X-ray, US, or so forth) monitoring the intravascular procedure. The guidewire 18 can exit one of the arms 32 for entry into the blood vessel V. The arms 32 each include the bent tip 40 to direct the exiting guidewire 18 approximately parallel to the blood vessel wall to further facilitate subintimal crossing entry. This embodiment does not include electromagnetics for directing the guidewire 18 into a selected one of the arms 32. Rather, in this embodiment the operator articulates (e.g. turns and/or twists) the guidewire 18 under X-ray or other imaging guidance to direct the tip of the guidewire into the desired arm. Alternatively, in some cases, the operator may not have a preference as to which arm the guidewire enters—in these cases, spacing the three arms 32 at equal circumferential angles (e.g., at 120° intervals) around the central axis CA of the catheter 12 serves to center the entry device 30 in the lumen of the blood vessel.

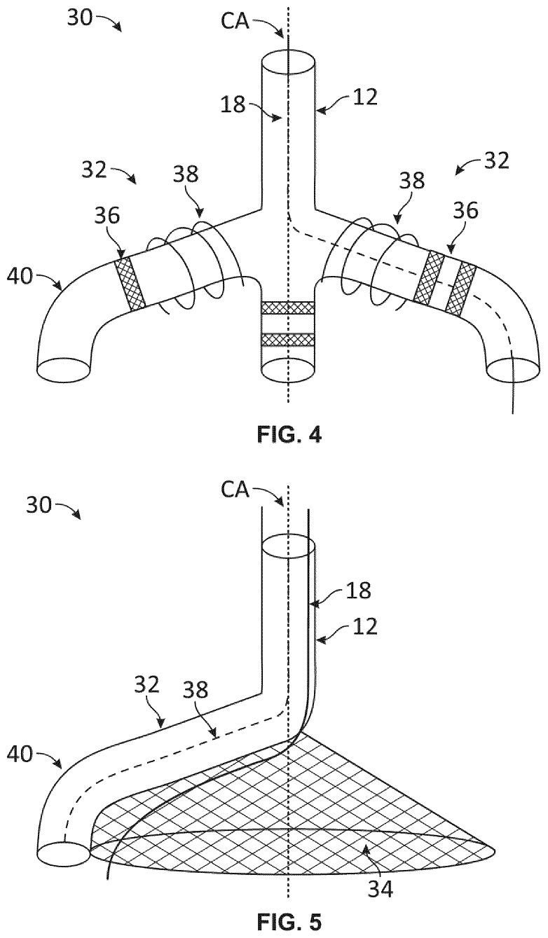

The embodiments of FIGS. 2 and 3 do not include a magnetic steering mechanism such as the electromagnets 38₁ and 38₂ of the embodiment of FIG. 1. With reference next to FIGS. 4-6, further embodiments that do include a magnetic steering mechanism are described.

FIG. 4 shows another embodiment of the entry device 30 that includes multiple arms 32 (three of which are shown in FIG. 4). This configuration is similar to that shown in FIG. 1, but has three arms 32 rather than two. As with the embodiment of FIG. 3, by having these arms at 120° intervals around the central axis CA, the entry device 30 is advantageously centered in the blood vessel. The entry device 30 of FIG. 4 includes a magnetic steering mechanism 38 disposed on each of the arms 32. The magnetic steering mechanism is configured to steer the tip 38 of the guidewire 18 as the tip exits the guidewire lumen 16 at the distal end of the catheter 12, similar to as was described for the embodiment of FIG. 1. In some embodiments, the magnetic steering mechanism 38 comprises at least one coil 38 surrounding at least one of the arms 32. As shown in FIG. 4, each of the arms 32 has a corresponding coil 38 wrapped around the arm. The coil(s) 38 suitably implement electromagnets (i.e., solenoids), which are selectively electrically energizable (e.g., via electrical leads passing through the catheter 12) to articulate the magnetic tip of the guidewire 18 into the selected arm when the guidewire 18 exits the lumen of the catheter 12. The arms 32 each include the bent tip 40 to direct the exiting guidewire 18 approximately parallel to the blood vessel wall to further facilitate subintimal crossing entry.

FIG. 5 shows another embodiment of the entry device 30 that includes one arm 32. In this embodiment, the guidewire 18 does not pass through the arm 32, but rather the guidewire 18 exits the end of the lumen of the catheter 12 into a blood vessel. In the absence of the entry device 30, this exit would typically be near the center of the blood vessel lumen, away from the blood vessel wall, thus, making subintimal crossing entry challenging. As shown in FIG. 5, the magnetic steering mechanism 38 of the entry device 30 comprises a magnetic rod 38 extending through the arm 32. The magnetic rod 38 may comprise, for example, an electromagnet or a permanent magnet. The tip of the guidewire 18 is steered along the arm 32 by magnetic attraction of the tip toward the magnetic rod 38. This draws the tip of the guidewire toward the blood vessel wall so as to facilitate subintimal crossing entry. The illustrative arm 32 again has a bend 40 at its tip to bias the guidewire to move parallel to the blood vessel wall as the guidewire approaches the subintimal crossing entry. Mesh 34 can also be included in this embodiment to further articulate the tip of the guidewire 18. In another example, the magnetic steering mechanism 38 is configured to magnetically attract the magnetic material comprising the tip of the guidewire 16 to steer the tip of the guidewire 18 as the tip exits the guidewire lumen 16 at the distal end of the catheter 12. In some examples, the arm 32 and the mesh 34 (not shown in FIG. 5) could deform the vessel V by pushing the vessel out to make it easier for the guidewire 18 to enter a sub-intimal space of the vessel V. Also, the guidewire 18 may be navigated towards a specific point inside the clot C and not necessarily towards the wall of the blood vessel V.

FIG. 6 shows another embodiment of the entry device 30 that operates similarly to the embodiment of FIG. 5, but which includes multiple arms 32 (six of which are shown in FIG. 6). By having the six arms at uniform 60° intervals, the entry device 30 is beneficially centered in the blood vessel lumen as described for the embodiments of FIGS. 3 and 4. (More generally, if there are N arms with N>2 then it is beneficial for centering to have the N arms uniformly spaced apart at 360°/N intervals). In the embodiment of FIG. 6, the magnetic steering mechanism 38 comprises a number of electromagnets 38 corresponding to the number arms 32, with each electromagnet 38 mounted on a corresponding arm 32. The electromagnets 38 are configured to be selectively electrically energized to steer the magnetic tip of the guidewire 18 as the tip exits the guidewire lumen 16 at the distal end of the catheter 12. To do so, each arm 32 has a lumen connected with the guidewire lumen 16 of the catheter 12 to receive the tip of the guidewire 18 as it exits the guidewire lumen 16 at the distal end of the catheter 12. The electromagnet(s) 38 are configured to steer the tip of the guidewire 18 as it exits from the guidewire lumen 16 into the lumen of a selected one of the arms 32 by selective electrical energization of the electromagnets 38. This configuration can advantageously provide finer circumferential steering—for example, if it is desired to steer the tip of the guidewire 18 to a subintimal entry at a circumferential position in-between two neighboring arms, then by selectively energizing the electromagnets 38 of those two arms the guidewire tip can be guided between the arms. The arms 32 again each include the bend 40 to direct the exiting guidewire 18 approximately parallel to the blood vessel wall to further facilitate subintimal crossing entry. Mesh 34 can also be included in this embodiment to further articulate the tip of the guidewire 18.

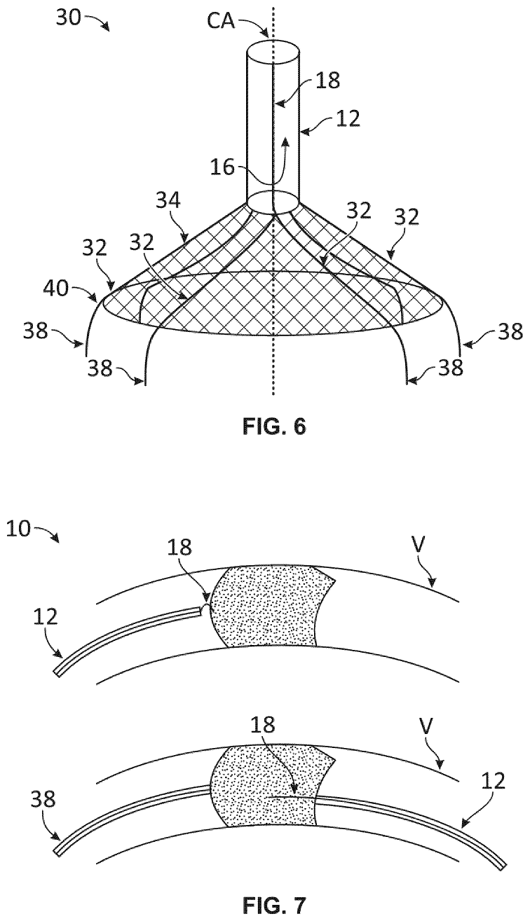

FIG. 7 shows another embodiment of the intravascular device 10. In some cases, if crossing the CTO from an antegrade approach is not possible, an interventionalist may, secondarily, try a retrograde approach. In this case, a magnetic force on the proximal (antegrade) side of the lesion or occlusion in the blood vessel V may be used to help pull the guidewire 18 through from the distal (retrograde) side of the lesion, as shown in FIG. 7. By pulling on the guidewire 18 across the lesion, rather than solely relying on pushing the intravascular device 10, the guidewire 18 can more easily advance through the lesion without buckling. The magnetic steering mechanism 38 may be centered in the lumen 16 of the catheter 12, or it may be positioned eccentrically. Eccentricity of the magnetic steering mechanism 38 may allow it to be positioned on the preferred side of the vessel V such that the guidewire 18 can be steered to avoid calcifications, for example.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. An intravascular therapy device, comprising:
a catheter having a guidewire lumen configured to pass a guidewire therethrough; and
at least one arm branching away from a distal end of the catheter at a nonzero angle respective to a central axis of the catheter, the at least one arm configured to steer a tip of the guidewire away from the central axis of the catheter as the tip of the guidewire exits the guidewire lumen at the distal end of the catheter.

2. The intravascular therapy device of claim 1, wherein the at least one arm includes:
a magnetic steering mechanism disposed on or in the at least one arm;
wherein the magnetic steering mechanism is configured to steer the tip of the guidewire as the tip of the guidewire exits the guidewire lumen at the distal end of the catheter.

3. The intravascular therapy device of claim 2, wherein the magnetic steering mechanism comprises a magnetic rod extending through the at least one arm, wherein the tip of the guidewire is steered along the at least one arm by magnetic attraction of the tip of the guidewire toward the magnetic rod.

4. The intravascular therapy device of claim 2, wherein the magnetic steering mechanism comprises at least one coil surrounding the at least one arm.

5. The intravascular therapy device of claim 2, wherein:
the at least one arm includes two or more arms; and
the magnetic steering mechanism comprises two or more electromagnets corresponding to the two or more arms with each electromagnet mounted on a corresponding arm;
wherein the two or more electromagnets are configured to be selectively electrically energized to steer the tip of the guidewire as the tip of the guidewire exits the guidewire lumen at the distal end of the catheter.

6. The intravascular therapy device of claim 5, wherein:
each arm has a lumen connected with the guidewire lumen of the catheter to receive the tip of the guidewire as the tip of the guidewire exits the guidewire lumen at the distal end of the catheter; and
the magnetic steering mechanism is configured to steer the tip of the guidewire as the tip of the guidewire exits from the guidewire lumen at the distal end of the catheter into the lumen of a selected one of the two or more arms by selective electrical energization of the two or more electromagnets.

7. The intravascular therapy device of claim 2, wherein the tip of the guidewire comprises a magnetic material, and the magnetic steering mechanism is configured to magnetically attract the magnetic material comprising the tip of the guidewire to steer the tip of the guidewire as the tip of the guidewire exits the guidewire lumen at the distal end of the catheter.

8. The intravascular therapy device of claim 1, wherein the at least one arm is elastic and configured to be stowed in the distal end of the catheter during delivery.

9. The intravascular therapy device of claim 1, further comprising:
a mesh attached to the at least one arm and configured to expand to bias the at least one arm into a position branching away from the distal end of the catheter at the nonzero angle respective to the central axis of the catheter.

10. The intravascular therapy device of claim 1, wherein each arm has a lumen connected with the guidewire lumen of the catheter to receive the tip of the guidewire as the tip of the guidewire exits the guidewire lumen at the distal end of the catheter and is steered away from the central axis of the catheter as the tip of the guidewire passes through the lumen of the arm.

11. An intravascular therapy method, comprising:
passing a guidewire through a guidewire lumen of a catheter; and
using at least one arm of a device, steering a tip of the guidewire away from a central axis of the catheter as the tip of the guidewire exits the guidewire lumen at a distal end of the catheter.

12. The intravascular therapy method of claim 1, wherein the steering includes:
magnetically steering the tip of the guidewire as the tip of the guidewire exits the guidewire lumen at the distal end of the catheter using a magnetic steering mechanism disposed on or in the at least one arm.

13. The intravascular therapy method of claim 12, wherein:
the at least one arm includes two or more arms;
the magnetic steering mechanism comprises two or more electromagnets corresponding to the two or more arms with each electromagnet mounted on a corresponding arm; and
the steering comprises selectively electrically energizing the two or more electromagnets to steer the tip of the guidewire as the tip of the guidewire exits the guidewire lumen at the distal end of the catheter.

14. The intravascular therapy method of claim 12, wherein the tip of the guidewire comprises a magnetic material, and the steering comprises steering the tip by magnetically attracting the magnetic material comprising the tip of the guidewire as the tip of the guidewire exits the guidewire lumen at the distal end of the catheter.

15. The intravascular therapy method of claim 11, further comprising:
moving the at least one arm into a position branching away from the distal end of the catheter using a mesh attached to the at least one arm and configured to expand to bias the at least one arm into the position branching away.
